(19) United States
(12) Patent Application Publication
(10) Pub. No.: US 2003/0177030 A1
(43) Pub. Date: Sep. 18, 2003

(54) PATIENT INFORMATION SYSTEM AND METHOD OF USING SAME

(75) Inventors: NICKIE C. TURNER, MUNCIE, IN (US); SHARON L. SCHEPP, MUNICE, IN (US)

Correspondence Address: MICHAEL B MCNEIL, LIELL MCNEIL & HARPER, 511 S MADISON STREET, P O BOX 2417, BLOOMINGTON, IN, 474022417

(73) Assignee: Michael B. McNeil

(21) Appl. No.: 09/442,343

(22) Filed: Nov. 17, 1999

(51) Int. Cl. G06F 17/60; H04L 9/00; H04L 9/32; G06F 11/30; G06F 12/14; G06F 15/173; A61B 5/00

(52) U.S. Cl. 705/2; 713/201; 713/202

(57) ABSTRACT

A patient information system comprises a computer server having a patient information forum for at least one patient. Each of the patient information forums contains information regarding an individual patient. A publicly accessible computer output device is networked to the computer server. Also networked to the computer server is a publicly accessible computer input device. A security subsystem is operably positioned between the patient information forum and the input device.

### TECHNICAL FIELD

The present invention relates generally to a patient information system, and more particularly to a patient information system that includes a publicly accessible computer server and an access limiting security subsystem.

### BACKGROUND ART

When a patient is admitted to a hospital or other treatment center there are often a number of family members that must be or desire to be contacted and/or informed regarding a course of treatment and related patient information, such as release dates, etc. Likewise, there are a number of third parties who contact caregivers with questions or concerns about a patient in their care. Physicians and other caregivers often spend an unnecessarily large part of their time attempting to contact these concerned parties or responding to the same questions and concerns multiple times for different factions of the group of persons interested in a particular patient. Time consuming problems also arise routinely when two different family members believe that they have received apparently conflicting information from the physician or other caregiver.

For instance, often more than one family member is involved in the decision making process regarding treatment of a patient. In these situations, information usually gets repeated to each family member separately due to the difficulties associated with organizing a meeting involving all family members and all treating caregivers. Often these family members forget, misinterpret or misunderstand what was said, increasing friction and leading to disagreements between family members. This inability to understand and assimilate the information provided can also lead to increased opportunities for litigation, which also dominates an unnecessarily large amount of physicians' and caregivers' time and financial resources.

A more efficient and consistent method of communication between caregivers and a patient's family members and friends is therefore desirable to allow caregivers to devote more of their time to patient treatment. This desire for a less time consuming and more reliable method of communication must be balanced, however, with the need to maintain confidentiality of a patient's medical records. In this respect, a communication system that meets the demands of caregivers and family members must also provide adequate security to protect a patient's confidential medical records and background information.

The present invention is directed to overcoming one or more of the problems set forth above and to providing a more efficient and consistent method of communication that adequately protects a patient's confidential records.

### DISCLOSURE OF THE INVENTION

In one aspect of the present invention, a patient information system comprises a computer server having a patient information forum for at least one patient. Each of the patient information forums contain information regarding an individual patient. A publicly accessible computer output device is networked to the computer server. Also networked to the computer server is a publicly accessible computer input device. A security subsystem is operably positioned between the patient information forum and the input device.

In another aspect of the present invention, a method of exchanging information regarding a patient first comprises the step of establishing a patient information forum for a particular patient on a computer server. Next, the computer server is networked into a publicly accessible computer network. Access to the patient information forum is then limited to a predetermined group of persons.

In yet another aspect of the present invention, a software program comprises a means for establishing a patient information forum on a publicly accessible computer network. A means for limiting access to the patient information forum to a predetermined group of persons is also included. Additionally, a means for posting information regarding a particular patient on the patient information forum is included.

### BRIEF DESCRIPTION OF THE DRAWINGS

FIG. 1 is a schematic view of a patient information system according to the present invention.

FIG. 2 is a pictorial representation of the various levels of security and privileges according to another aspect of the present invention.

FIG. 3 is a diagrammatic representation of the data input structure of the present invention.

FIG. 4 is an example of an administration control screen according to the present invention.

FIG. 5 is an example of a patient detail screen according to the present invention.

FIG. 6 is a diagrammatic representation of the message posting structure for responsible and non-responsible parties according to the present invention.

FIG. 7 is a diagrammatic representation of the message posting structure for administration and staff members according to the present invention.

FIG. 8 is an example of a sign-on screen according to the present invention.

FIG. 9 is an example screen within a patient information forum according to the present invention.

### BEST MODE OF CARRYING OUT THE INVENTION

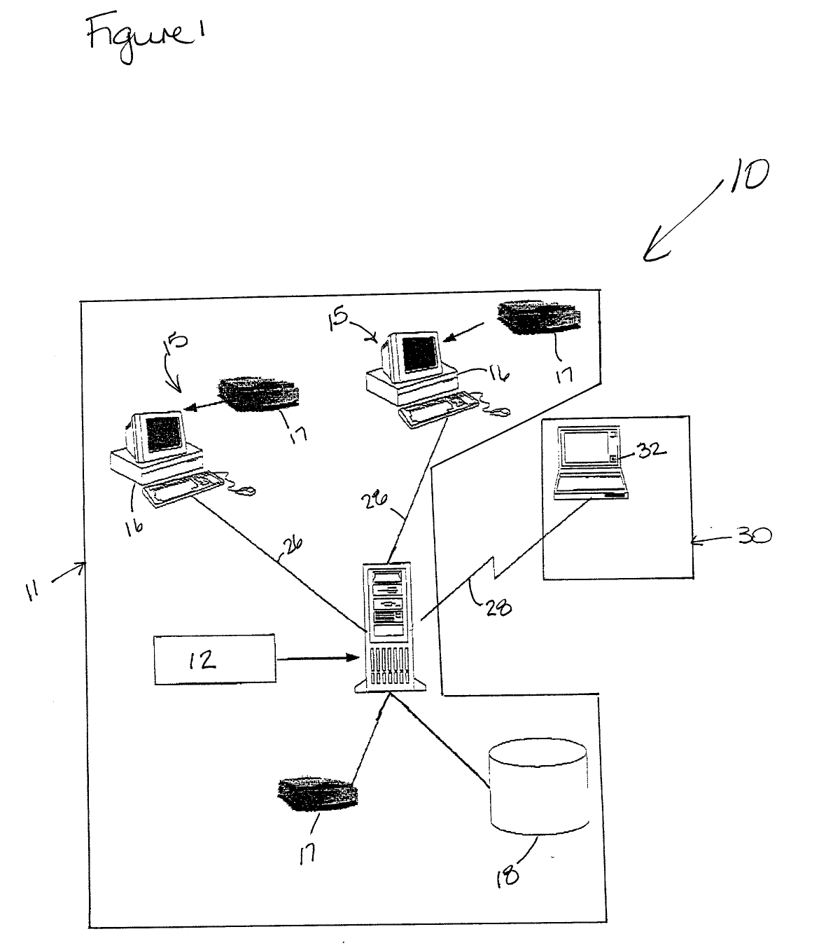

Referring now to FIG. 1 there is shown a schematic view of a patient information system 10 according to the present invention. Information system 10 is preferably composed of four elements, including the environment, the file system, the languages and detailed processing used and the security system. A computer server 12, which is preferably located at a suitable place such as within treatment center 11, controls information system 10 and serves as an integral component of the environment of information system 10. The environment including computer server 12 is preferably a PC network, however, other systems, such as a legacy mainframe system, could be used. A PC network is preferable because the PC allows for quick and easy deployment and it also allows for very cost effective graphics and graphical interfaces. While in most instances, treatment center 11 will be a hospital, it should be appreciated that the present invention could also find use in other facilities where patients remain institutionalized for treatment, such as nursing homes, outpatient clinics, and in-patient clinics, etc.

Information system 10 utilizes an indexing file system, such as VBISAM sold by Software Source, Inc., for information management. While VBISAM is preferable, it should be appreciated that other indexing file systems, such as those sold under the Trademarks ACCESS, INFORMIX and ORACLE, could alternatively be used. The programming language used for the main program modules of information system 10 is preferably Microsoft Visual Basic. The help tips and file location searches are preferably programmed in another programming language, such as Microsoft C. While these languages are preferable, it should be appreciated that the various modules and components of information system 10 could be programmed in virtually any language with varying degrees of processing efficiency.

Returning now to the hardware for information system 10, computer server 12 is connected to a variety of on-site workstations 15 via internal communication lines 26. These workstations 15 are preferably placed in various locations throughout treatment center 11. A number of workstations 15 should be placed in locations where concerned parties, such as responsible family members, non-responsible family members and friends of the patient will be able to access information system 10. Examples of these publicly accessible locations would be the waiting room, patient rooms, the ICU area, the CCU area and the emergency room. In addition to those workstations 15 contemplated for concerned party use, a number of workstations 15 should preferably be placed in locations such that caregivers and other employees of treatment center 11 would be the primary users. These locations could include the admitting department, the billing department, the nurses station, the physician's office and workstation and the pharmacy. While a number of desirable locations for workstations 15 have been described, it should be appreciated that the present invention contemplates workstations that could be located in any convenient or necessary location throughout treatment center 11. The appropriate number and availability of workstations 15 for the facility would be influenced in part by such factors as the nature and size of the facility itself.

Workstations 15 are preferably kiosks that include one or more computer terminals 16 connected to server 12. Each terminal 16 should include a computer output device, such as a computer monitor, and a computer input device. While a keyboard and a mouse will be sufficient input devices in most instances, it is preferable that some terminals include at least one computer interface for users with special needs. Examples of these special needs interfaces include those for the visually impaired, a touchscreen interface or a voice recognition interface. By incorporating at least one of these special needs interfaces in terminals 16 connected to server 12, the present invention will be more accommodating to those persons who might otherwise have difficulty accessing information system 10, such as the elderly or those with a physical disability. A document scanner 17 is also preferably included in one or more of the workstations 15 that are primarily available for caregiver use. Inclusion of document scanner 17 can allow caregivers to scan such things as test results or x-rays for review by responsible parties or other caregivers.

In addition to document scanner 17, a data storage device 18 is also connected to server 12. Data storage device 18 is included to store patient information that is transmitted via information system 10. Once stored, this information can be accessed at a later time by caregivers, responsible parties, or the patient himself. In addition to test results or x-rays, data storage device 18 is intended to store messages posted regarding the patient and the course of treatment in case they are needed in the future. For example, information stored in data storage device 18 could be used by treatment center 11 or a caregiver in the event that litigation regarding the course of treatment for a particular patient is initiated at some time in the future.

In addition to those terminals connected to server 12 by internal communication lines 26, information system 10 is designed to be accessed from off-site locations 30 via a publicly accessible computer network such as the Internet. For instance, a concerned party could access information system 10 from an off-site location 30. Server 12, which is also an Internet server, could be networked into a publicly accessible computer network to allow third party individuals to access information system 10 from locations outside of treatment center 10. A computer input and output device 32 in the concerned party's home or office could be networked to server 12 via publicly accessible Internet communication lines 28 to allow the user to access information system 10. This would allow the concerned party to access current information and/or leave questions at any time of day or night from any off-site location. The ability to access information system 10 over the Internet would find particular usefulness for those concerned parties residing too far away from treatment center 11 to be present during the patient's treatment, or for those who were otherwise unable to be physically present when caregivers are available for questions.

Likewise, a physician could access information system 10 from his or her office 30 by connecting office computer 32 to server 12 via Internet communication lines 28. In this manner, a physician could answer questions and update information from his or her off-site office 30 without having to attempt to personally communicate with all of the patient's concerned parties. Similarly, a hospitalist, a physician who is primarily based in treatment center 11, could utilize information system 10 to access records and other information from a referring physician. For instance, the hospitalist and the referring physician could establish a dialogue in information system 10 via Internet communication lines 28 to exchange information regarding the patient. A direct dial-up access could be substituted for an internet connection. The hospitalist would therefore be spared from traveling between treatment center 11 and the referring physician's office 30 to secure needed treatment information.

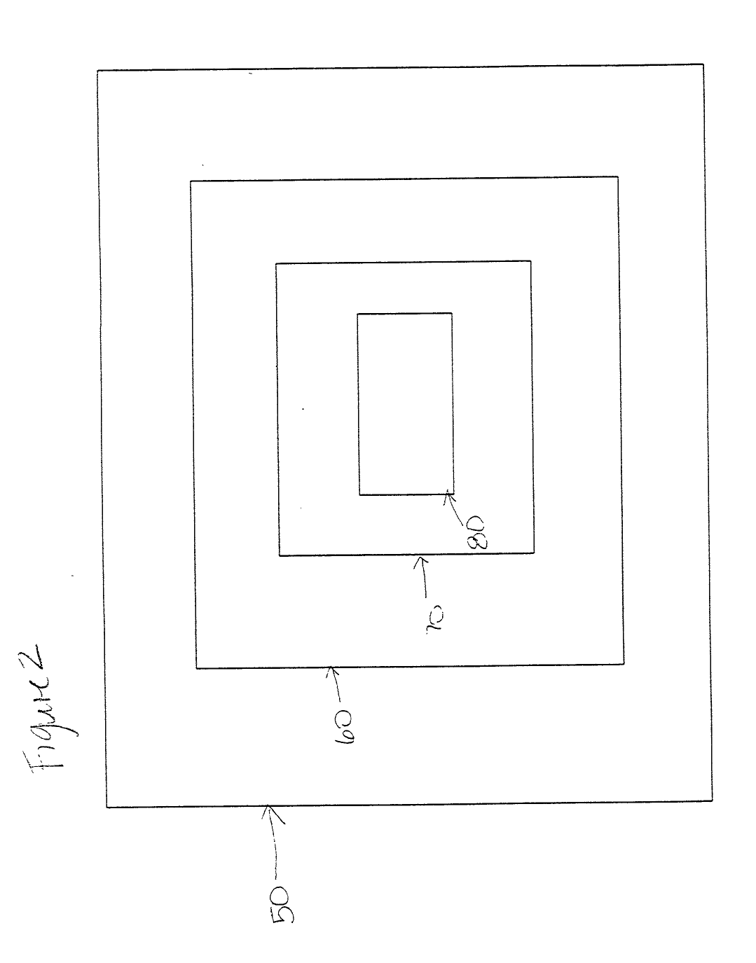

As previously indicated, the desire for a more efficient and accessible system for physician and concerned party communication must be balanced with the need to maintain adequate security of the patient's medical records and other confidential information. Therefore, a security system, preferably a multi-level security clearance system, is one of the key elements of information system 10. Referring now to FIG. 2 there is shown a schematic view of the security clearance levels created by information system 10. The security levels 50-80 are operably positioned between the input device and a patient information forum that exists for the patient. Clearance through the security levels is preferably obtained utilizing passwords assigned to a user at admission, in the case of a patient, or the initiation of employment, in the case of a caregiver or staff member of treatment center 11. For example, a treating physician may have a single password that allows total access to all patients in his or her care, etc. However, it should be appreciated that other security means could be substituted, such as security keycards. Additionally, while four security levels have been illustrated, it should be appreciated that this number could vary to better meet the needs of the individual treatment center 11.

The four predetermined security levels illustrated in FIG. 2 determine the amount of access that users will be granted upon entry into information system 10. Once a patient is admitted to treatment center 11 for treatment of his or her particular ailment, one or more passwords are created to be used in conjunction with the patient's name. The patient, guardian or a person having power of attorney then determines who will be given the passwords, thus allowing the patient or other person with authority to determine who has access to information, and how much access that person should be allowed. For instance, a user who is permitted access into a first security level 50 might be a non-responsible third party. Members of this group could include anyone not having any legal responsibility for the patient, such as friends or distant relatives of the patient. Those gaining access to this security level might have the ability to leave questions for the patient and access answers to these questions. In addition, they might have general access to non-confidential discharge information and non-confidential background information regarding the treating physician. This party may not, however, be able to access more confidential information such as patient diagnosis information or treatment records. Those skilled in the art will appreciate that some minimal information such as discharge data may be available to non-responsible parties but more detailed information, such as post-discharge instructions, would not be available to non-responsible persons.

A second security level 60 could be created to allow responsible parties such as a person holding a power of attorney, the patient's spouse, parent or guardian, or the patient himself to have a greater degree of access to information relating to the patient. In this instance, the user may be able to access background information and medical records in addition to having the ability to post questions to caregivers or the patient and to read postings regarding the patient. This level might also include links to other databases, such as WebMD, Dr. Koops website and others relating to general information regarding medical information and/or a particular ailment or condition.

A third security level 70 could be created for users who are employed in the administrative departments of treatment center 11, such as the billing department or the business office. In addition, the person or persons responsible for maintaining information system 10 and its security could be included in this group of users. These users could sign in from the same screen as responsible or non-responsible parties, however, they could select a function key to notify information system 10 that they were neither a responsible nor a non-responsible party. These users would not only be able to access medical records and to post and answer questions, but, in addition, they could input and edit information such as billing/payment information, dietary restrictions, insurance and employment information and other information of that nature. These users might not, however, be able to update or change information relating to medication dosages or other information regarding the course of treatment currently being pursued.

Finally, a fourth security level 80 might be created for physicians, nurses or other caregivers. Once again, users from this group could enter information system 10 through the same sign in screen as responsible or non-responsible parties, however, selection of a function key could notify information system 10 that they were either an administrative employee or a caregiver. Proper entry into this level could allow the user full ability to access information and update records, in addition to answering questions left by concerned parties. This security level is contemplated to allow the user to have total access to the patient's information. Once again, it should be appreciated that while four security levels have been illustrated, this number could be reduced or increased to meet the needs of the treatment center and patients. For example, first security level 50 and second security level 60 could be merged into a single security level for all concerned parties. Alternatively, the number of security levels could be increased by further dividing fourth security level 80 into multiple levels for each class of caregiver such that there would exist separate security levels for nurses, pharmacists and physicians. In this manner, the present invention can be adapted to meet the needs of the particular treatment center. In another security aspect, the present invention preferably creates an audit trail by recording time, identity and other information to track each incident in which the system is accessed. In addition, the system also preferably logs all unsuccessful attempts to access the information system.

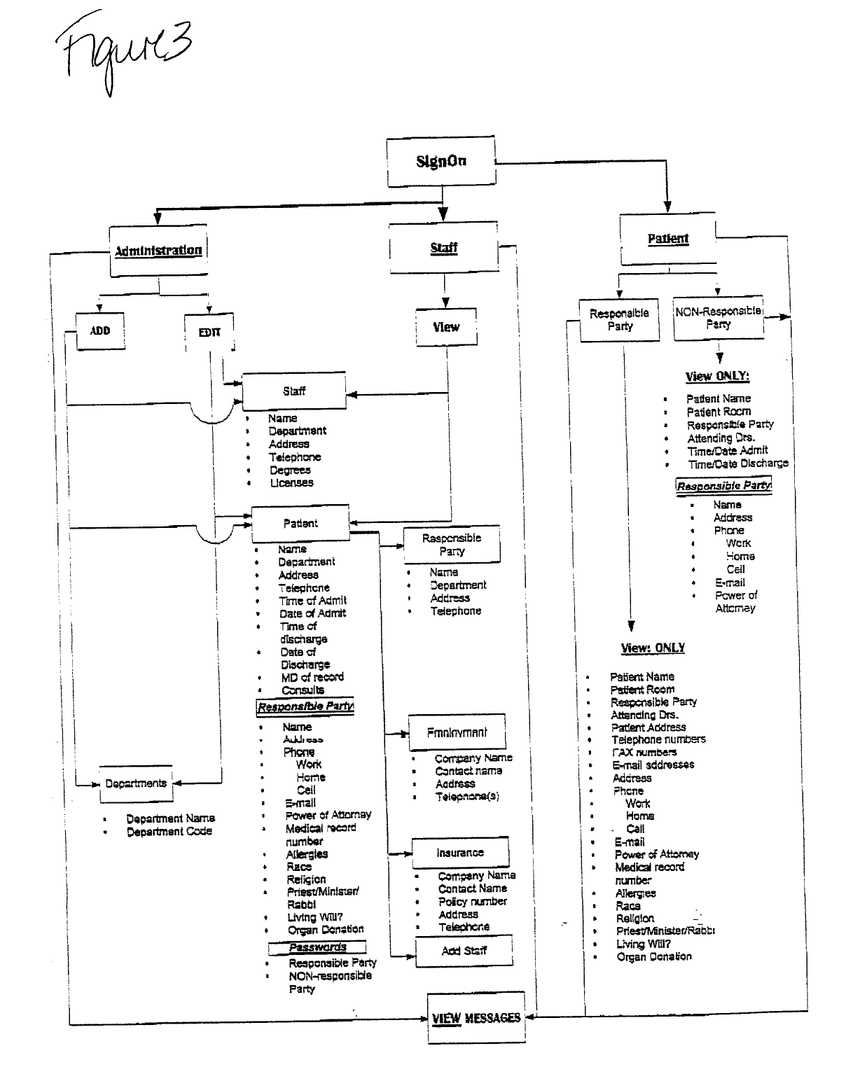

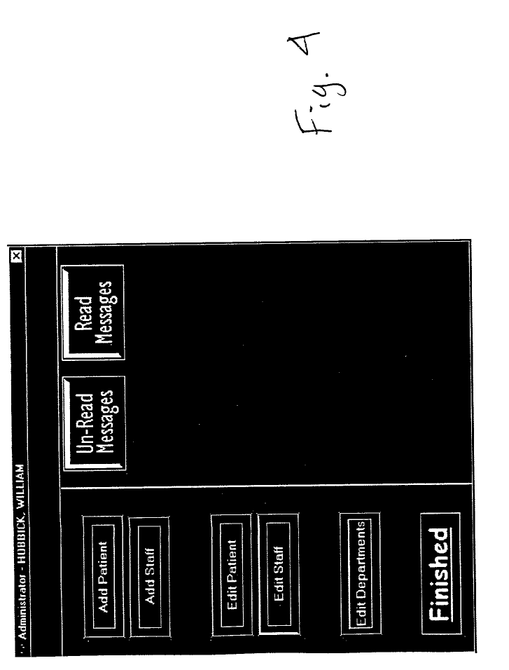

Referring now to FIG. 3 there is shown the data input structure of information system 10. FIGS. 3-5 further illustrate options available to a user once information system 10 has been accessed, depending on the level of security to which that person is entitled. For instance, a user that is classified as an administrator, such as an employee working in the billing department or the business office, could add or edit information regarding staff, patients or departments. A party signing on to information system 10 under this classification would encounter an administrator control screen (FIG. 4) that includes a number of options to allow the administrator to continuously update information within information system 10. From this screen, the administrator could add new patients or staff, edit patient information or staff information, edit any information associated with the various departments of treatment center 11, and read all read and unread messages that have been posted to the system and intended for the particular administrative person currently signed on. For instance, by selecting the add patient option, an administrator could input the passwords unique to the particular patient as well as other initial data such as social security number, date of admission, address or date of birth. In addition, once a patient is added into the system via this screen, information system 10 could assign a unique ID number to the patient or forum, preferably composed of the current year, month and day and a seven digit patient number.

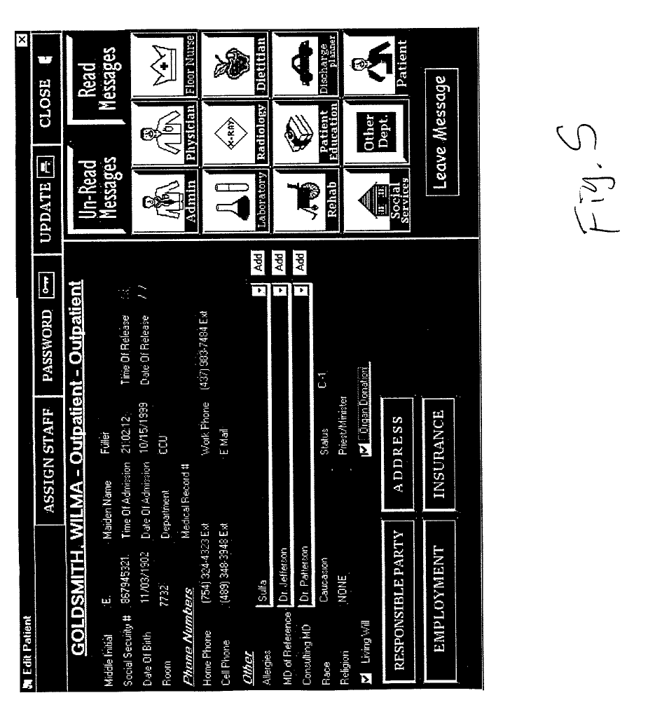

In addition to data input from the add patient screen, more detailed information, such as allergies, reference doctor and consulting doctor could be added to the patient information, preferably by selection of a detail icon located on the add patient screen. The patient detail screen (FIG. 5) could also display the patient's message status and allow the administrator to view existing messages or send messages. In addition, the patient's responsible party, employment and insurance data could be entered from this screen and stored as part of the patient's master record.

Returning to FIG. 4, the administrator could select the edit patient option to access the same screen accessed from the add patient option. Selection of the edit patient option would result in a select patient screen that would allow any number of letters of a patient last name to initiate a search of the database and produce a list of all patients meeting the search criteria. Therefore, to access patient information through the edit patient option, the patient should have been previously entered via the add patient option, or they will not appear in the results of the search. Use of the edit patient option also differs from use of the add patient option in that it allows changing of the patient's responsible and non-responsible party passwords, but not the patient name, social security number or date and time of admission. These four pieces of information are considered the indexed file system's key, and can therefore only be entered or altered from the add patient option.

Returning once again to FIG. 4, selection of the add staff or edit staff options from the administration control screen would allow the administrator to perform similar functions as those performed using the add patient and edit patient options. Those persons categorized as staff members could include various caregivers, technicians, discharge planners and radiologists, as well as any employee of treatment center 11 who would have reason to use information system 10. The administrator could input such data as date of employment, address, phone numbers, schooling and social security number using the add staff option. Upon adding the staff member, information system 10 could assign a unique ID number, similar to that of the unique patient ID number, consisting of the year, month and day and a seven-digit number. Selection of the edit staff option would produce the same screens as the add staff option except the administrator would be presented with a change password option. Additionally, a staff selection screen, similar to the patient selection screen produced when the edit patient option is chosen, would be presented to allow the administrator to search the database for those names meeting a given name selection criteria.

Finally, an administrator could change information regarding the various departments of treatment center 11 by selecting the edit departments option. From this option, the administrator could print all the department codes and descriptions, list the department codes, display the department codes and descriptions, edit the department codes and descriptions, add department codes and descriptions or delete department codes and descriptions. This would allow the administrator to maintain an accurate list of departments and their descriptions as the needs and services of treatment center 11 change and grow.

Returning now to FIG. 3, a user accessing information system 10 as a staff member would be able to view, but not edit or add, such information as staff data and certain patient information, including responsible party information, employment and insurance information. In addition to the above abilities, staff members would also be able to review messages regarding treatment of the patient. Finally, those persons accessing information system 10 as a patient, a responsible party, or a non-responsible party would be able to view patient information as well as review messages regarding the patient's treatment. As illustrated in FIG. 3, a responsible party user could access more detailed information regarding the patient, such as race, religious preference and allergies, than could a non-responsible party.

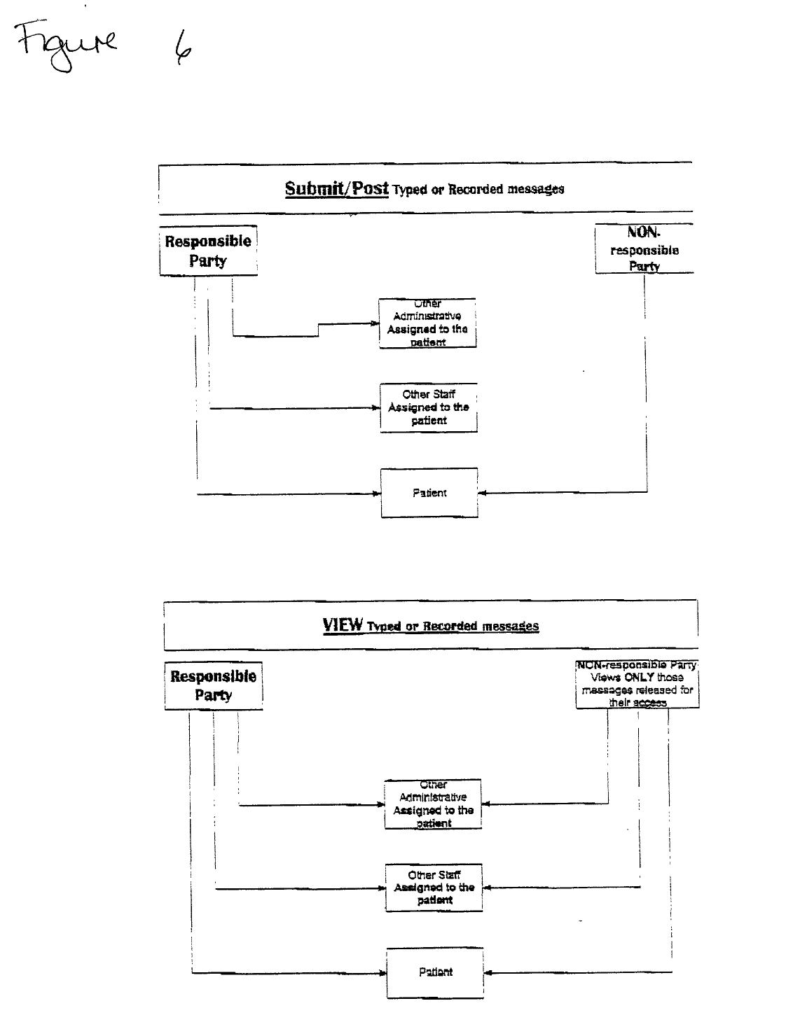

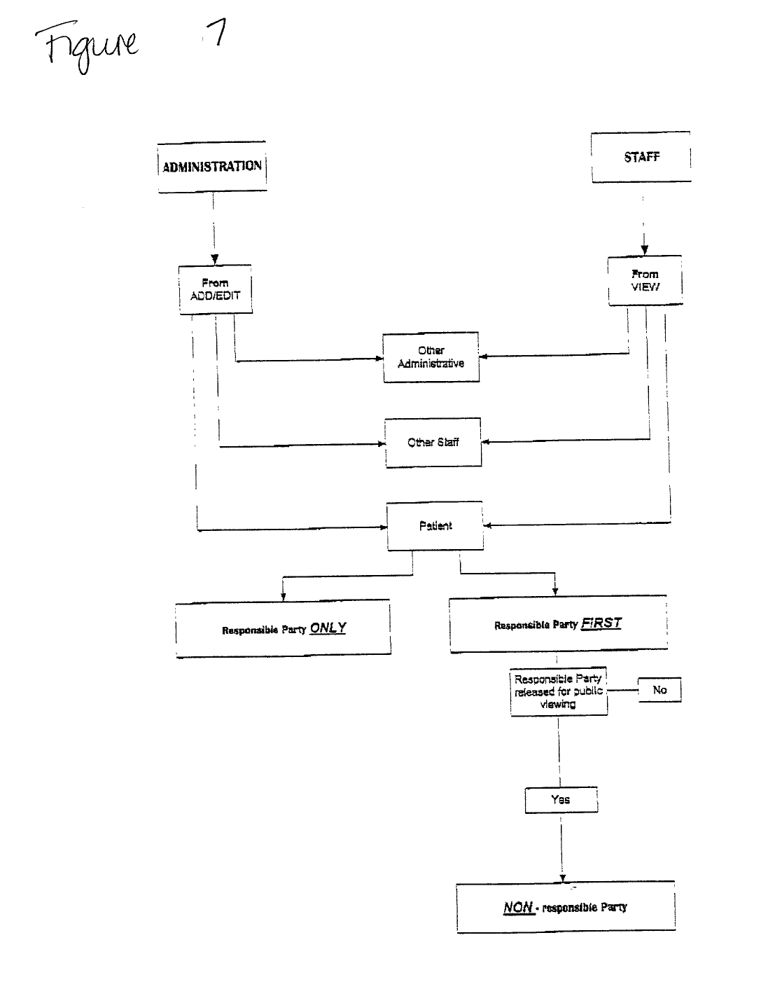

Referring now to FIGS. 6-7 there are shown message posting structures for responsible and non-responsible parties as well as for administration and staff members. As illustrated in FIG. 6, a responsible party could submit messages to both administrators and staff assigned to treat the patient, as well as posting messages to the patient himself and/or the responsible party. Similarly, the responsible party could read or review messages posted from administrative employees, staff members or the patient. While a non-responsible party could read or review messages posted by any of these parties, as illustrated in FIG. 6, the non-responsible party would preferably only have the ability to leave messages for the patient. Preferably, messages for the responsible party are automatically released to non-responsible parties after being read by the responsible party. Private messages for the responsible party are not released to non-responsible parties. When either the responsible or non-responsible party opts to review a message, information system 10 could generate a list of messages, including date and time posted and format of the message. In other words, a symbol could be included with the message information to alert the concerned party as to whether the message is a text or audio message. In addition, in the case of audio messages, it is preferable that the length of the message is provided for the user so he or she may determine if the message should be reviewed presently or some time later when they have more time.

The ability for a patient or his responsible party to post and receive messages regarding a patient's treatment allows information system 10 to be a powerful tool for patient treatment by providing those concerned with an increased amount of information. This increased information can allow patients and responsible parties to make more informed decisions regarding treatment in addition to reducing the anxiety that is often associated with medical treatment. Additionally, because information from the caregiver is posted a single time, all concerned parties can view the same information, which can reduce the friction associated with information that has been repeated multiple times and possibly misinterpreted or misunderstood.

In addition to the above benefits, concerned parties now have the ability to post a question for the caregiver regarding treatment at any time, rather than waiting for the opportunity for a face to face consultation. When a patient is being cared for in a large treatment center, responsible parties usually can only speak with the doctor when he or she is doing their rounds. This time is often short, and even more often is at a very early hour of the day. In addition to the concerns of parties not able to meet with the caregivers at these times, responsible parties may forget to ask questions during these consultations or may think of new questions after the consultation is over. When a patient or responsible party feels that not enough information was given them, or that their concerns were not adequately addressed, they are more likely to initiate litigation if treatment is not as successful as they desired. However, information system 10 can help lessen this threat of litigation because a patient or a responsible party can post a question or concern for the caregiver as it develops, and receive a follow-up without having to wait for another short consultation. In this manner, the likelihood of litigation can be reduced, thereby reducing the amount of time physicians must devote to lawsuits rather than patient treatment.

Returning now to FIG. 7, administrative employees and staff members could preferably post and receive messages from other administrative employees, other staff members and the patient himself. When posting a message for the patient, the administrative employee or staff member could signify whether the message will be accessible to responsible parties only, or merely to responsible parties first. In the later case, the responsible party would have the option to release the message to non-responsible parties after viewing its content. In this manner, the administrator or staff member could release sensitive information only to those persons responsible for the patient thus enhancing the privacy or security of information system 10.

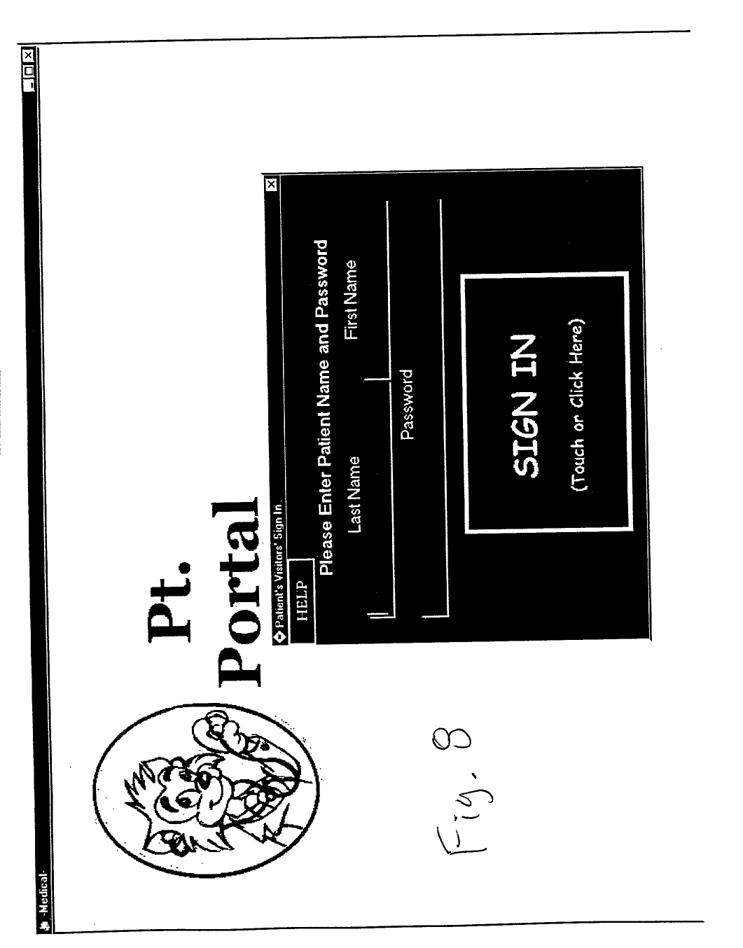

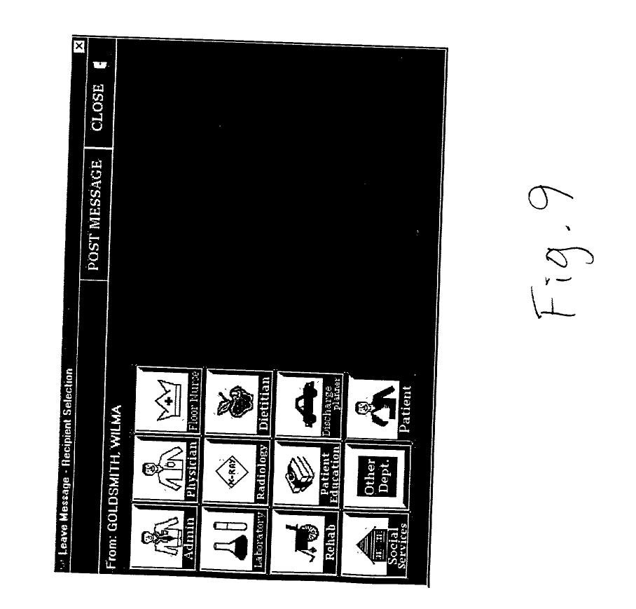

Referring now to FIGS. 8-9, there are shown additional screens that a user might encounter when accessing information system 10. These Figures will be used to help illustrate how a concerned party or caregiver would navigate through information system 10. It should be appreciated that these screens are being used for illustrative purposes only, and are not meant to limit the scope of the present invention.

When a patient is admitted to treatment center 11 for treatment of an ailment, a patient information forum 20 is created on computer server 12 that contains information regarding the patient. Patient information forum 20 is subdivided into a plurality of levels corresponding to security levels 50-80. The patient then selects, or is assigned, at least one password that can be distributed to a predetermined group of persons to allow them to access patient information forum 20. Preferably, the patient should select two passwords, a first for himself and his responsible parties, and a second for all other persons who may wish to see and communicate with the patient and the attending staff. To maximize the number of passwords that information system 10 could accept, these passwords are preferably case sensitive and can include not only letters and numbers, but symbols and spaces as well. By making the passwords case sensitive and allowing spaces, it should also be appreciated that system security could be enhanced by requiring persons to not only have the correct characters, but also the correct case and spacing.

Once passwords are selected by the patient, and once the patient or responsible party has tested the passwords to ensure that they function adequately, they can be distributed to the corresponding family members and friends. If one of these third party individuals desires entry into information system 10, and more particularly to patient information forum 20, that user must first connect to computer server 12. This can be done either by activating a terminal at one of the computer workstations 15 located at treatment center 11 or by networking with server 12 via the unique Internet address assigned to patient information system 10. Upon connection to server 12, the user should encounter an identification screen (FIG. 8). To access patient information forum 20, the user will usually be required to input certain information, such as the patient's name, both first and last, and a password. This password will determine the level of access entitled to the user and will allow the user to pass through at least one of the security levels operably positioned between the computer input device and patient information forum 20.

Once the information is entered in the identification screen, server 12 will evaluate the password to determine what level of access, if any, the user will be granted. If the patient name and password are not accepted, an error message should appear to alert the user of the invalid password or patient name. If the patient name and password are accepted, the user is granted entry to a level of patient information forum 20. Once in patient information forum 20, the user can read messages regarding treatment of the patient's ailment or leave questions regarding the same for a caregiver. As shown in FIG. 9, a bulletin board is created in patient information forum 20 which serves as a location for dialogue. It is here that the user can access updates on the patient and the patient's ailment. This is also where the user could leave questions or concerns for caregivers. As illustrated, a link is created for a number of departments within treatment center 11. If the user desires information from a particular department, he or she would follow the appropriate link to access a database having information regarding the patient's ailment. By separating the information by source, it is also possible to deny access to postings from certain departments depending on the level of access allowed to the particular user. For instance, the user's password might entitle him or her to access information posted by the patient, the floor nurse and the physician but not from the other sources such as the billing department or the social worker.

By following the appropriate link, the user could access various postings from each source. This would be done by using a mouse to click on the appropriate button, or alternatively by using a predetermined keyboard shortcut, touching areas on a screen or using voice commands. After reading or listening to the information posted, the user would then have the opportunity to respond to a particular notice, or alternatively, the user would be presented with an opportunity to leave questions for the caregiver or patient, and possibly to review questions and answers already posted. Likewise, at appropriate times during the course of treatment, the caregiver could add updates to patient information forum 20 regarding the patient's ailment and the course of treatment, such as test results. Once the user has read all of the desired updates and/or left any questions or concerns for the caregiver, patient information forum 20 could be exited by following the appropriate link, thus preventing subsequent unauthorized users from accessing confidential patient information.

The present invention is intended to increase the quality, efficiency and consistency of communication between caregivers and responsible or concerned parties relating to a particular patient. By utilizing the present invention, physicians and other caregivers will no longer have to spend valuable time trying to locate these parties only to have to repeat the same facts and answer the same questions to a number of different parties. Instead, the caregivers can post updates on a virtual bulletin board that is accessible only by those persons authorized by the treatment center or the patient. Likewise, a concerned party can leave questions for the caregiver that can be directly addressed without the need for actual, real-time interaction. The present invention can therefore not only increase the quality of information that is exchanged between the caregiver and the patient or his responsible party, but can even help reduce the amount of litigation that results from misunderstood or misinterpreted information. In this manner, the present invention can allow caregivers to spend more of their time treating patients instead of locating concerned parties or responding to litigation.

It should be understood that the above description is intended for illustrative purposes only, and is not intended to limit the scope of the present invention in any way. For instance, while the present invention has been illustrated using passwords as a means of obtaining access to different levels of secured sites, it should be appreciated that other means could be used, such as a security system based on personal identification numbers or keycards. Additionally, while a number of example screens have been shown to illustrate the various features of the information system of the present invention, it should be appreciated that these are merely samples and are not meant to limit the invention in any way. Further, while four levels of secured access have been disclosed, it should be appreciated that a different number of levels could be employed to better meet the needs of the facility. Additionally, while the present invention has been described for use in a hospital setting, it should be appreciated that it would find application in any facility in which patients are treated, such as nursing homes or assisted living communities, outpatient clinics, etc. Further, the present invention could be used to keep third parties informed of the condition of persons being detained for a non-medical reason, such as persons incarcerated in a prison. Thus, those skilled in the art will appreciate the various modifications could be made to the disclosed embodiments without departing from the intended scope of the present invention, which is defined in terms of the claims set forth below.

1. A patient information system comprising:
a computer server having a patient information forum for at least one patient, and each said patient information forum containing information regarding an individual patient;
a publicly accessible computer output device networked to said computer server;
a publicly accessible computer input device networked to said computer server; and
a security subsystem operably positioned between said patient information forum and said input device.

2. The patient information system of claim 1 wherein said patient information forum is subdivided into a plurality of levels, and a different portion of said patient information forum being available at different ones of said plurality of levels; and
said security subsystem being operable to limit access to said patient information forum to a predetermined group of persons, and limit access to different ones of said levels to subsets of said predetermined group of persons.

3. The patient information system of claim 2 wherein said predetermined group of persons includes third party individuals identified by a particular patient to which said patient information forum relates, and said third party individuals include relatives of said particular patient.

4. The patient information system of claim 1 wherein a particular patient has an ailment; and
said patient information forum for said particular patient includes a link to a database having information regarding said ailment.

5. The patient information system of claim 1 wherein said patient information forum includes a location for dialogue.

6. The patient information system of claim 1 wherein said computer server is an Internet server; and
said patient information system has an Internet address.

7. The patient information system of claim 1 wherein said security subsystem includes an assignment of passwords to a predetermined group of persons that are approved for access to said patient information forum of a particular patient.

8. A method of exchanging information regarding a patient comprising the steps of:
establishing a patient information forum for a particular patient on a computer server;
networking said computer server into a publicly accessible computer network; and
limiting access to said patient information forum to a predetermined group of persons.

9. The method of claim 8 wherein said establishing step includes a step of dividing said patient information forum into a plurality of levels; and
said limiting access step includes a step of limiting subsets of said predetermined group of persons to different ones of said levels.

10. The method of claim 8 further comprising the steps of:
identifying said predetermined group of persons; and
assigning a password to each person in said predetermined group of persons.

11. The method of claim 8 wherein said establishing step includes a step of providing a location for dialogue in said patient information forum.

12. The method of claim 8 wherein said particular patient has an ailment; and
said establishing step includes a step of providing a link in said patient information forum to a database having information regarding said ailment.

13. The method of claim 8 wherein said networking step includes a step of providing a patient information system with a Internet address; and
networking said computer server to at least one Internet server.

14. The method of claim 8 further comprising the steps of:
networking a special needs computer to said computer server; and
providing said special needs computer with at least one of a visually impaired computer interface, a touchscreen computer interface and a voice recognition computer interface.

15. The method of claim 8 further comprising a step of providing said patient information forum with a bulletin board location having new information added to said patient information forum.

16. A software program comprising:
means for establishing a patient information forum on a publicly accessible computer network;
means for limiting access to said patient information forum to a predetermined group of persons; and
means for posting information regarding a particular patient on said patient information forum.

17. The software program of claim 16 wherein said means for limiting includes:
means for requesting a password; and
means for evaluating a password.

18. The software program of claim 16 further comprising means for enabling a dialogue among said predetermined group of persons.

19. The software program of claim 16 wherein a particular patient has an ailment; and
means for linking said patient information forum to a database having information regarding said ailment.

20. The software program of claim 17 wherein said means for limiting access includes a means for creating gradations of access dependent upon evaluation of said password.
